(19) United States
(12) Patent Application Publication
(10) Pub. No.: US 2024/0342724 A1
(43) Pub. Date: Oct. 17, 2024

(54) DETECTION CHIP AND PREPARATION METHOD THEREFOR, AND DETECTION METHOD

(71) Applicants: BEIJING BOE TECHNOLOGY DEVELOPMENT CO., LTD., Beijing (CN); BOE TECHNOLOGY GROUP CO., LTD., Beijing (CN)

(72) Inventors: Houqian GAO, Beijing (CN); Zijian ZHAO, Beijing (CN); Ding DING, Beijing (CN); Shinying LAU, Beijing (CN)

(21) Appl. No.: 18/580,314

(22) PCT Filed: Sep. 7, 2022
(86) PCT No.: PCT/CN2022/117509

§ 371 Date: Jan. 18, 2024

(30) Foreign Application Priority Data
Oct. 29, 2021 (WO) PCT/CN2021/127599

(51) Int. Cl. B01L 3/00

(52) U.S. Cl. CPC B01L 3/5085; B01L 2300/0893; B01L 2300/12; B01L 2300/161

(57) ABSTRACT

A detection chip and a manufacturing method thereof, and a detection method are provided. The detection chip includes: a base substrate, and a detection layer, provided on the base substrate and including a plurality of detection holes. An inner wall of each of at least part of detection holes among the plurality of detection holes is hydrophilic, and a contact angle of the inner wall is within 30 degrees.

### CROSS-REFERENCE TO RELATED APPLICATION

The present application claims the priority to International Application No. PCT/CN2021/127599, filed on Oct. 29, 2021, the entire disclosure of which is incorporated herein by reference as portion of the present application.

### TECHNICAL FIELD

Embodiments of the present disclosure relate to a detection chip and a manufacturing method thereof, and a detection method.

### BACKGROUND

DNA sequencing technology is one of the most commonly used technical means in molecular biology-related research, which has promoted the rapid development of this field to a certain extent. Currently, a sequencing chip may be used to complete a process of sequencing reaction and detection. During the process, the structure and total number of independent partition units of the sequencing chip directly affect the sequencing result.

### SUMMARY

At least one embodiment of the disclosure provides a detection chip, the detection chip comprises: a base substrate, and a detection layer, provided on the base substrate and comprising a plurality of detection holes, wherein an inner wall of each of at least part of detection holes among the plurality of detection holes is hydrophilic, and a contact angle of the inner wall is within 30 degrees.

For example, in the detection chip provided by at least one embodiment of the disclosure, a surface of the detection layer far away from the base substrate is hydrophobic relative to the inner wall of the at least part of detection holes, and a contact angle of the surface is 80-150 degrees, or the surface of the detection layer far away from the base substrate is provided with a microstructure, and a contact angle of at least a surface of the microstructure far away from the base substrate is 80-150 degrees.

For example, in the detection chip provided by at least one embodiment of the disclosure, a surface roughness Ra of the microstructure is 250 nm-300 nm.

For example, in the detection chip provided by at least one embodiment of the disclosure, the microstructure comprises a carbonized adhesive layer or an ITO layer doped with ZnO.

For example, in the detection chip provided by at least one embodiment of the disclosure, the detection layer comprises a silicon oxide layer.

For example, in the detection chip provided by at least one embodiment of the disclosure, a diameter of each detection hole among the plurality of detection holes is 0.2 microns-3.0 microns, a distance between adjacent detection holes is 0.5 microns-2.5 microns, and a distance between centers of adjacent detection holes is 0.8 microns-5.0 microns.

For example, in the detection chip provided by at least one embodiment of the disclosure, a depth of each detection hole among the plurality of detection holes is 0.5 microns-3.0 microns.

For example, in the detection chip provided by at least one embodiment of the disclosure, a surface of the detection layer far away from the base substrate and/or the inner wall of at least one detection hole among the plurality of detection holes are provided with a metal element.

For example, in the detection chip provided by at least one embodiment of the disclosure, the inner wall of each among the plurality of detection holes comprises a side wall and a bottom wall, and at least one of the side wall and the bottom wall is provided with an uneven structure.

For example, in the detection chip provided by at least one embodiment of the disclosure, a cross-sectional shape, parallel to the base substrate, of at least one detection hole among the plurality of detection holes is a circle, and a cross-sectional shape, perpendicular to the base substrate, of at least one detection hole among the plurality of detection holes is a rectangle or a trapezoid.

For example, in the detection chip provided by at least one embodiment of the disclosure, a surface of the detection layer far away from the base substrate is provided with a microstructure, and the microstructure comprises a base layer and a hydrophobic layer provided on a side of the base layer far away from the detection layer; and the base layer is an ITO layer, and the hydrophobic layer is an ITO layer doped with ZnO.

For example, in the detection chip provided by at least one embodiment of the disclosure, a diameter of each detection hole among the plurality of detection holes is 2.0 microns-3.0 microns, and a distance between adjacent detection holes is 0.5 microns-1.5 microns.

For example, in the detection chip provided by at least one embodiment of the disclosure, a depth of each detection hole among the plurality of detection holes is 0.5 microns-1.5 microns, and a thickness of the microstructure is 0.2 microns-0.8 microns.

For example, in the detection chip provided by at least one embodiment of the disclosure, a surface of the detection layer far away from the base substrate is provided with a microstructure, and the microstructure comprises a base layer and a hydrophobic layer provided on a side of the base layer far away from the detection layer, and the base layer is a metal layer, and the hydrophobic layer is a carbonized adhesive layer.

For example, in the detection chip provided by at least one embodiment of the disclosure, the metal layer is an Al layer or a multilayer structure comprising an Al layer.

For example, in the detection chip provided by at least one embodiment of the disclosure, the hydrophobic layer comprises a plurality of annular portions respectively surrounding the plurality of detection holes, the plurality of annular portions form a concave portion between every four adjacent detection holes, and a material of the hydrophobic layer does not exist in the concave portion.

For example, in the detection chip provided by at least one embodiment of the disclosure, a diameter of each detection hole among the plurality of detection holes is 1.3 microns-2.5 microns, and a distance between adjacent detection holes is 0.5 microns-2.0 microns.

For example, in the detection chip provided by at least one embodiment of the disclosure, a depth of each detection hole among the plurality of detection holes is 0.5 microns-1.5 microns, and a thickness of the microstructure is 0.2 microns-1.0 micron.

For example, the detection chip provided by at least one embodiment of the disclosure further comprises: a base adhesive layer, provided between the base substrate and the detection layer.

For example, the detection chip provided by at least one embodiment of the disclosure further comprises: a first protective layer, comprising silicon dioxide and provided between the base adhesive layer and the detection layer, and a second protective layer, comprising silicon dioxide and provided on a side of the detection layer far away from the base substrate, wherein the detection layer comprises a photoresist material.

For example, in the detection chip provided by at least one embodiment of the disclosure, a diameter of each detection hole among the plurality of detection holes is 0.2 microns-2.2 microns, and a depth of each detection hole among the plurality of detection holes is 0.5 microns-1.5 microns.

For example, in the detection chip provided by at least one embodiment of the disclosure, a cross-sectional shape, perpendicular to the base substrate, of at least one detection hole among the plurality of detection holes comprises two arc-shaped edges protruding toward each other.

For example, in the detection chip provided by at least one embodiment of the disclosure, a first cross-section, parallel to the base substrate, of the at least one detection hole has a first diameter, a second cross-section, parallel to the base substrate, of the at least one detection hole has a second diameter, and a third cross-section, parallel to the base substrate, of the at least one detection hole has a third diameter, the second cross-section is located on a side of the first cross-section far away from the base substrate, and the third cross-section is located on a side of the second cross-section far away from the base substrate, and the second diameter is smaller than the first diameter and the third diameter.

For example, in the detection chip provided by at least one embodiment of the disclosure, the detection layer comprises a first sub-detection layer and a second sub-detection layer provided on a side of the first sub-detection layer far away from the base substrate, the first sub-detection layer is an adhesive layer, and the second sub-detection layer is a silicon oxide layer, and the detection hole comprises a first sub-detection hole provided in the first sub-detection layer and a second sub-detection hole provided in the second sub-detection layer, and the first sub-detection hole and the second sub-detection hole are interpenetrated with each other.

For example, in the detection chip provided by at least one embodiment of the disclosure, an orthographic projection of the second sub-detection hole on the base substrate is located inside an orthographic projection of the first sub-detection hole on the base substrate.

For example, in the detection chip provided by at least one embodiment of the disclosure, a diameter of the first sub-detection hole is 1.0 micron-2.5 microns, and a depth of the first sub-detection hole is 1.0 micron-1.8 microns, and a diameter of the second sub-detection hole is 0.6 microns-1.8 microns, and a depth of the second sub-detection hole is 0.4 microns-0.8 microns.

For example, in the detection chip provided by at least one embodiment of the disclosure, a cross-sectional shape, parallel to the base substrate, of each of the at least part of detection holes among the plurality of detection holes is a circle, and a cross-sectional shape, perpendicular to the base substrate, of each of at least part of detection holes among the plurality of detection holes is an inverted trapezoid.

For example, in the detection chip provided by at least one embodiment of the disclosure, a length of a long side of the inverted trapezoid is 1.2 microns-2.2 microns, a length of a short side of the inverted trapezoid is 0.5 microns-1.8 microns, and a height of the inverted trapezoid is 1.0 micron-1.8 microns.

For example, in the detection chip provided by at least one embodiment of the disclosure, a cross-sectional shape, parallel to the base substrate, of each of the at least part of detection holes among the plurality of detection holes is a circle, and a cross-sectional shape, perpendicular to the base substrate, of each of the at least part of detection holes among the plurality of detection holes is a rectangle.

For example, in the detection chip provided by at least one embodiment of the disclosure, a diameter of each of the at least part of detection holes among the plurality of detection holes is 1.0 micron-2.2 microns, and a depth of each of the at least part of detection holes among the plurality of detection holes is 1.0 micron-1.8 microns.

At least on embodiment of the disclosure further provides a manufacturing method of a detection chip, the manufacturing method comprises: providing a base substrate; and forming a detection layer on the base substrate, wherein the detection layer comprises a plurality of detection holes, an inner wall of each of at least part of detection holes among the plurality of detection holes is hydrophilic, and a contact angle of the inner wall is within 30 degrees.

For example, manufacturing method provided by at least one embodiment of the disclosure further comprises: performing hydrophobic treatment on a surface of the detection layer far away from the base substrate to enable a contact angle of the surface to be 80-150 degrees; or forming a microstructure on the surface of the detection layer far away from the base substrate, wherein a contact angle of at least a surface of the microstructure far away from the base substrate is 80-150 degrees.

For example, in the manufacturing method provided by at least one embodiment of the disclosure, forming the detection layer and the microstructure comprises: forming a detection material layer on the base substrate; forming a base material layer on a side of the detection material layer far away from the base substrate; forming a hydrophobic material layer on a side of the base material layer far away from the base substrate; forming a photoresist pattern on a side of the hydrophobic material layer far away from the base substrate; and patterning the hydrophobic material layer, the base material layer and the detection material layer by using the photoresist pattern as a mask to form the detection layer and the microstructure comprising a base layer and a hydrophobic layer, the base layer is an ITO layer, and the hydrophobic layer is an ITO layer doped with ZnO.

For example, in the manufacturing method provided by at least one embodiment of the disclosure, forming the detection layer and the microstructure comprises: forming a detection material layer on the base substrate; forming a base material layer on a side of the detection material layer far away from the base substrate; forming a photoresist pattern on a side of the base material layer far away from the base substrate; patterning the base material layer and the detection material layer by using the photoresist pattern as a mask to form the detection layer and a base layer; and performing carbonization treatment on the photoresist pattern to form the microstructure.

At least on embodiment of the disclosure further provides a detection method using the detection chip as described above, the detection method comprises: preparing a reaction wash solution; preparing a reaction mother solution; preparing a plurality of groups of reaction solution by using the reaction mother solution, wherein the plurality of groups of reaction solution comprise a first group of reaction solution; introducing the reaction wash solution into the detection chip and lowering a temperature of the detection chip to 3° C.-6° C.; taking a fluorescence image; introducing the first group of reaction solution into the detection chip and raising the temperature of the detection chip to 60° C.-70° C.; and taking a fluorescence image.

For example, in the detection method provided by at least one embodiment of the disclosure, the plurality of groups of reaction solution further comprise a second group of reaction solution, and the detection method further comprises: introducing the reaction wash solution into the detection chip and lowering the temperature of the detection chip to 3° C.-6° C.; taking a fluorescence image; introducing the second group of reaction solution into the detection chip and raising the temperature of the detection chip to 60° C.-70° C.; and taking a fluorescence image.

For example, in the detection method provided by at least one embodiment of the disclosure, the reaction wash solution comprises Tris-HCl, (NH4) 2SO4, KCl, MgSO4 and Tween®20.

For example, in the detection method provided by at least one embodiment of the disclosure, the reaction mother solution comprises Tris-HCl, (NH4) 2SO4, KCl, MgSO4, Tween®20, enzyme and CIP.

### BRIEF DESCRIPTION OF DRAWINGS

In order to clearly illustrate the technical solutions of the embodiments of the present disclosure, the drawings of the embodiments will be briefly described. It should be noted that the described drawings in the following are only related to some embodiments of the present disclosure and thus are not limitative of the present disclosure. For those skilled in the art, other related drawings can be obtained according to these drawings without inventive work.

FIG. 1A is a schematic plan view of a detection chip provided by at least one embodiment of the present disclosure;

FIG. 1B is a schematic cross-sectional view of the detection chip in FIG. 1A along a line A-A;

FIG. 1C is another schematic cross-sectional view of the detection chip in FIG. 1A along the line A-A;

FIG. 2 is a schematic cross-sectional view of another detection chip provided by at least one embodiment of the present disclosure;

FIG. 3A and FIG. 3B are scanning electron micrographs of a planar structure and a cross-sectional structure of the detection chip in FIG. 2;

FIG. 4A is a schematic plan view of another detection chip provided by at least one embodiment of the present disclosure;

FIG. 4B is a schematic cross-sectional view of the detection chip in FIG. 4A along a line B-B;

FIG. 5A and FIG. 5B are scanning electron micrographs of a planar structure and a cross-sectional structure of the detection chip in FIG. 4A and FIG. 4B;

FIG. 6 is a schematic cross-sectional view of another detection chip provided by at least one embodiment of the present disclosure;

FIG. 7A and FIG. 7B are scanning electron micrographs of two cross-sectional structures of the detection chip in FIG. 6;

FIG. 8 is a schematic cross-sectional view of yet another detection chip provided by at least one embodiment of the present disclosure;

FIG. 9A and FIG. 9B are scanning electron micrographs of a cross-sectional structure and a three-dimensional structure of the detection chip in FIG. 8;

FIG. 10A is a schematic plan view of yet another detection chip provided by at least one embodiment of the present disclosure;

FIG. 10B is a schematic cross-sectional view of the detection chip in FIG. 10A along a line C-C;

FIG. 11A and FIG. 11B are scanning electron micrographs of a planar structure and a cross-sectional structure of the detection chip in FIG. 10A and FIG. 10B;

FIG. 12A is a schematic plan view of still another detection chip provided by at least one embodiment of the present disclosure;

FIG. 12B is a schematic cross-sectional view of the detection chip in FIG. 12A along a line D-D;

FIG. 13A and FIG. 13B are scanning electron micrographs of a planar structure and a cross-sectional structure of the detection chip in FIG. 12A and FIG. 12B;

FIG. 14 is another schematic plan view of the detection chip provided by at least one embodiment of the present disclosure;

FIG. 15A is yet another schematic plan view of the detection chip provided by at least one embodiment of the present disclosure;

FIG. 15B is still another schematic plan view of the detection chip provided by at least one embodiment of the present disclosure;

FIG. 16A is a schematic cross-sectional view of still another detection chip provided by at least one embodiment of the present disclosure;

FIG. 16B is a schematic plan view of a cover plate in still another detection chip provided by at least one embodiment of the present disclosure;

FIG. 17 is a diagram of AFM test results of the detection chip provided by at least one embodiment of the present disclosure; and

FIG. 18 is a diagram of EDS test results of the detection chip provided by at least one embodiment of the present disclosure.

### DETAILED DESCRIPTION

In order to make objects, technical details and advantages of the embodiments of the present disclosure apparent, the technical solutions of the embodiments will be described in a clearly and fully understandable way in connection with the drawings related to the embodiments of the disclosure. Apparently, the described embodiments are just a part but not all of the embodiments of the present disclosure. Based on the described embodiments herein, those skilled in the art can obtain other embodiment(s), without any inventive work, which should be within the scope of the present disclosure.

Unless otherwise defined, all the technical and scientific terms used herein have the same meanings as commonly understood by one of ordinary skill in the art to which the present disclosure belongs. The terms “first,” “second,” etc., which are used in the description and the claims of the present disclosure, are not intended to indicate any sequence, amount or importance, but distinguish various components. The terms “comprise,” “comprising,” “include,” “including,” etc., are intended to specify that the elements or the objects stated before these terms encompass the elements or the objects and equivalents thereof listed after these terms, but do not preclude the other elements or objects. The phrases “connect”, “connected”, etc., are not intended to define a physical connection or mechanical connection, but may include an electrical connection, directly or indirectly. “On,” “under,” “left,” “right” or the like are only used to indicate relative position relationship, and when the position of the object which is described is changed, the relative position relationship may be changed accordingly.

In a process of DNA sequencing using a detection chip, in order to enable the sequencing reaction of each DNA unit to proceed independently and smoothly, for example, hundreds of millions of independent reaction partition units need to be formed in the detection chip to support the immobilization of DNA molecules, achieve high-throughput sequencing, and prevent detection crosstalk between adjacent reaction partition units. In this regard, the structure of the independent reaction partition unit of the sequencing chip needs to be designed to meet the above-mentioned requirements. During the process, how to form a structurally stable and high-throughput sequencing chip by a low-cost means is a problem being faced by those skilled in the art.

At least one embodiment of the present disclosure provides a detection chip and a manufacturing method thereof, and a detection method. The detection chip includes a base substrate and a detection layer, and the detection layer is provided on the base substrate and includes a plurality of detection holes. An inner wall of each of at least part of detection holes among the plurality of detection holes is hydrophilic, and the contact angle of the inner wall is within 30 degrees.

The above-mentioned detection chip provided by the embodiments of the present disclosure simply may form the plurality of detection holes in the detection layer through a semiconductor manufacturing process to achieve the purpose of high throughput. The total number of the detection holes may reach hundreds of millions, and the inner wall of each of at least part of the detection holes is hydrophilic, so the substance to be detected and the detection reagent are more likely to gather in the detection holes, and crosstalk is not easily formed between adjacent detection holes, thereby improving the detection accuracy.

In the following, the detection chip and the manufacturing method thereof, and the detection method provided by the embodiments of the present disclosure are described in detail through several specific embodiments.

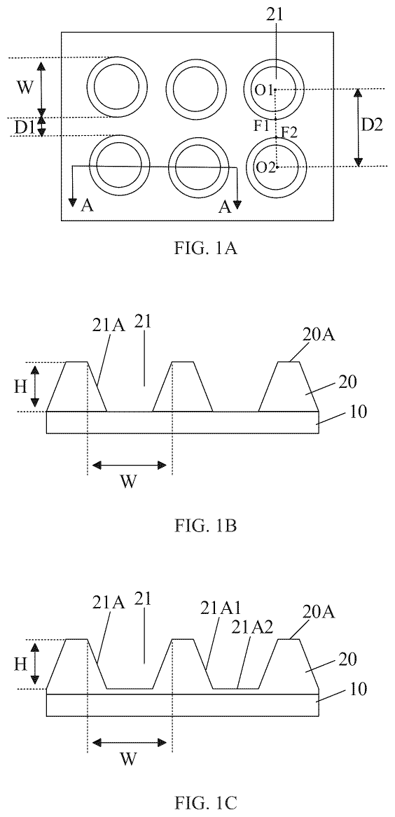

At least one embodiment of the present disclosure provides the detection chip, FIG. 1A illustrates a schematic plan view of the detection chip, and FIG. 1B illustrates a schematic cross-sectional view of the detection chip in FIG. 1A along a line A-A.

As illustrated in FIG. 1A and FIG. 1B, the detection chip includes a base substrate 10 and a detection layer 20. The detection layer 20 is provided on the base substrate 10, and includes a plurality of detection holes 21, the inner wall 21A of each of at least part of detection holes 21 (for example, all detection holes 21) among the plurality of detection holes 21 is hydrophilic, and the contact angle of the inner wall is within 30 degrees, such as within 20 degrees, such as within 10 degrees, such as within 5 degrees, such as between 2 degrees and 5 degrees, so that the inner wall 21A has a higher hydrophilicity, which is beneficial for the substance to be detected and detection reagent to gather in the detection holes 21, and crosstalk is not easily formed between adjacent detection holes 21, thereby improving the detection accuracy of the detection chip.

For example, in some embodiments, the inner wall 21A of the detection hole 20 having a higher hydrophilicity is the side wall of the detection hole 20; and in other embodiments, the inner wall 21A of the detection hole 20 having a higher hydrophilicity includes the side wall and the bottom wall of the detection hole 20.

For example, in some embodiments, as illustrated in FIG. 1B, a surface 20A of the detection layer 20 far away from the base substrate 10 is hydrophobic relative to the inner wall 21A of the above-mentioned at least part of detection holes 20, that is, the surface 20A is more hydrophobic and less hydrophilic than the inner wall 21A of the above-mentioned at least part of detection holes 20. For example, the contact angle of the surface 20A is 80-150 degrees, such as 90 degrees, 100 degrees, 110 degrees, 120 degrees, 130 degrees, 140 degrees or 150 degrees, etc.

For example, in some embodiments, as illustrated in FIG. 1B, the detection hole 21 in the detection layer 20 is a through hole penetrating the detection layer 20. In this case, the detection hole 21 exposes the structure below the detection layer 20, such as the base substrate 10. For example, in some other embodiments, as illustrated in FIG. 1C, the detection hole 21 in the detection layer 20 is a blind hole that does not penetrate the detection layer 20, in this case, the detection hole 21 does not expose the structure below the detection layer 20.

For example, in some embodiments, the detection layer of the detection chip is formed through a patterning process using a metal mask. In this case, during the manufacturing process, the metal mask is formed on the detection layer, and the metal mask needs to be removed after the patterning process is completed. However, in practice, the metal mask may not be completely removed.

For example, in some embodiments, the surface 20A of the detection layer far away from the base substrate 10 and/or the inner wall 21A of at least one detection hole 21 among the plurality of detection holes 21 have a metal element, and the metal element is, for example, a residue of the metal mask remaining on the surface 20A and/or the inner wall 21A of the detection hole 21 during the manufacturing process.

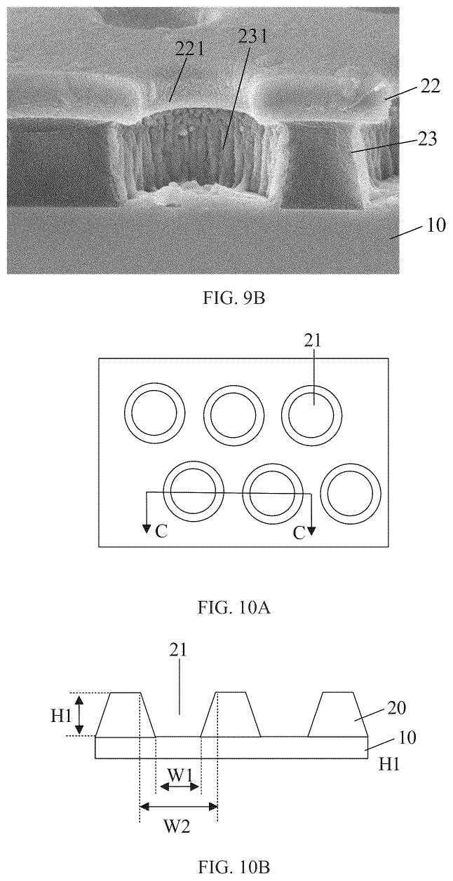

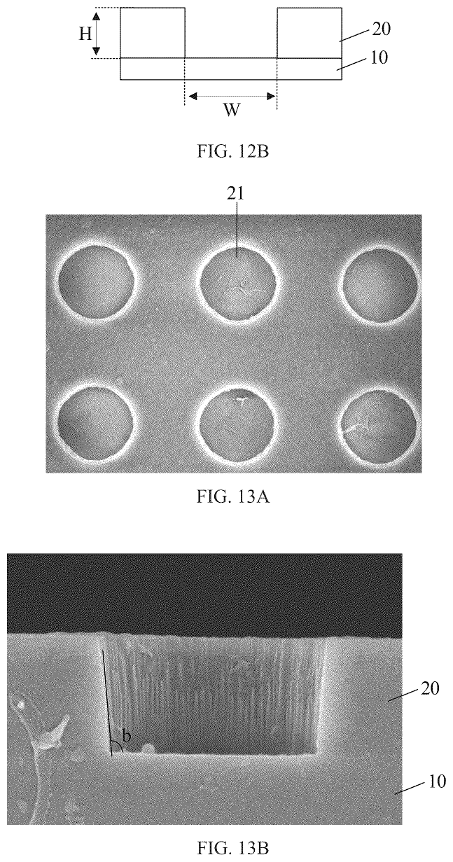

For example, in some embodiments, as illustrated in FIG. 1C, the inner wall 21A of each detection hole 21 includes a side wall 21A1 and a bottom wall 21A2, and at least one of the side wall 21A1 and the bottom wall 21A2 is provided with an uneven structure, such as fish scales or burrs described later, as illustrated in FIG. 5B, FIG. 9B, FIG. 11B, etc.

Alternatively, in some other embodiments, as illustrated in FIG. 2, the surface of the detection layer 20 far away from the base substrate 10 is provided with a microstructure 30, and the contact angle of at least the surface 30A of the microstructure 30 far away from the base substrate 10 is 80-150 degrees, such as 90 degrees, 100 degrees, 110 degrees, 120 degrees, 130 degrees, 140 degrees or 150 degrees, etc., thereby utilizing the microstructure 30 to achieve a hydrophobic effect.

In the embodiments of the present disclosure, the contact angle is a parameter of the wettability of a liquid on a surface of a solid material, and refers to an included angle from a solid-liquid interface, through the interior of the liquid, to a gas-liquid interface at the three-phase junction of solid, liquid, and gas. The smaller the included angle, the easier it is for the liquid to wet the solid, indicating a better wettability.

For example, in some embodiments, the surface roughness Ra of the microstructure 30 is 250 nm-300 nm, such as 260 nm, 270 nm, 280 nm or 290 nm, etc.

For example, in some embodiments, the microstructure 30 includes a carbonized adhesive layer or an ITO layer doped with ZnO, or the like. It is obtained from the test that the contact angle of the carbonized adhesive layer is about 150 degrees, and the contact angle of the ITO layer doped with ZnO is about 80-100 degrees, which is much larger than the contact angle of the side wall 21A of the detection hole 21, thereby facilitating the flow of the substance to be detected and the detection reagent injected into the microstructure 30 into the detection hole 21; moreover, the carbonized adhesive layer or the ITO layer doped with ZnO can be formed on the detection layer 20 by a simple semiconductor manufacturing process, which is beneficial to reduce the manufacturing difficulty and the manufacturing cost of the detection chip.

For example, in some embodiments, the detection layer 20 includes a silicon oxide layer, that is, the material of the detection layer 20 is an oxide of silicon, namely SiₓO_{y}, where x and y may be any suitable values. The oxide of silicon itself has a certain degree of hydrophilicity. For example, in some embodiments, the silicon oxide layer may also be hydrophilized to have a better hydrophilicity.

For example, in some embodiments, as illustrated in FIG. 1A and FIG. 1B, the diameter W of each detection hole 20 among the plurality of detection holes 20 is 0.2 microns-3.0 microns, such as 0.5 microns, 1.0 micron, 1.5 microns, 2.0 microns, 2.5 microns or 3.0 microns, etc.; the distance DI between adjacent detection holes 21 is 0.5 microns-2.5 microns, for example 1.0 micron, 1.5 microns, 2.0 microns or 2.5 microns, etc.; and the distance D2 between centers O1 and O2 of adjacent detection holes 21 is 0.8 microns-5.0 microns, such as 1.0 micron-4.0 microns, such as 1.5 microns-3.0 microns, such as 1.5 microns, 1.7 microns, 1.85 microns, 1.95 microns, 2.0 microns, 2.5 microns or 2.6 microns, etc.

In the embodiments of the present disclosure, the above-mentioned distance D1 between adjacent detection holes 21 is the shortest distance between the edges of the adjacent detection holes 21, or a distance between two intersection points F1 and F2 of a line connecting the centers O1 and O2 of the adjacent detection holes 21 and the edges of the adjacent detection holes 21.

For example, in some embodiments, the depth H of each detection hole 21 among the plurality of detection holes 21 is 0.5 microns-3.0 microns, such as 1.0 micron, 1.5 microns, 2.0 microns or 2.5 microns, etc., to fully accommodate the substance to be detected and the detection reagent.

In the embodiments of the present disclosure, in the case where the dimension of the detection hole 21 is too large, the scanning speed will slow down when scanning and detecting the plurality of detection holes 21, which will affect the detection efficiency, and the effective data obtained in the unit area will be reduced, thereby affecting the detection throughput; and in the case where the dimension of the detection hole 21 is too small, it may be difficult to achieve the detection due to the limited resolution of the detector, resulting in missed detection. In the case where the diameter of the detection hole 21, the distance between adjacent detection holes 21 and the like meet the above-mentioned dimension requirements, the requirements for detection throughput and detection resolution can be balanced.

For example, in some embodiments, an adapter primer (not illustrated in the figures) is provided in the detection hole 21, and the adapter primer is connected to the inner wall of the detection hole 21 by a covalent bond. By connecting the adapter primer to a surface of the hydrophilic layer 30 by a covalent bond, the adapter primer can be more firmly fixed in the detection hole 21 for the subsequent reaction and detection steps. For example, the adapter primer is a segment of DNA for linking to DNA fragments to be detected. For example, in some examples, the covalent bond is —CO—NH—.

For example, in some embodiments, the detection chip further includes a cover layer (not illustrated in the figures), and the cover layer is provided on a side of the adapter primer far away from the base substrate 10. The cover layer can protect the adapter primer and help achieve long-term preservation of the detection chip, for example, it can be preserved for one year or even longer. For example, the material of the cover layer includes a water-soluble polymer, such as a copolymer of N-(5-azidoacetamidopentyl) acrylamide and acrylamide, etc., so that the cover layer can be removed by a simple water washing step to expose the adapter primer.

For example, in some embodiments, as illustrated in FIG. 1A, a cross-sectional shape, parallel to the base substrate 10, of each of at least part of detection holes 21 (for example, all the detection holes 21) among the plurality of detection holes 21 is a circle; and as illustrated in FIG. 1B, a cross-sectional shape, perpendicular to the base substrate 10, of each of at least part of detection holes 21 among the plurality of detection holes 21 is a rectangle or a trapezoid, and FIG. 1B illustrates an example in which the cross-sectional shape is a trapezoid.

For example, in the embodiments of the present disclosure, the cross-sectional shape of a structure being of a figure means that the outline of the cross-sectional shape of the structure roughly follows the figure; due to the process and other reasons, the cross- sectional shape of the structure is often not strictly in the shape of the figure, but in the shape of the deformation of the figure, such as some edges are inclined, offset or curved, etc.

For example, FIG. 2 illustrates a schematic cross-sectional view of the detection chip provided by at least one embodiment of the present disclosure. As illustrated in FIG. 2, in the present embodiment, the surface of the detection layer 20 far away from the base substrate 10 is provided with a microstructure 30, and the microstructure 30 includes a base layer 31 and a hydrophobic layer 32 provided on a side of the base layer 31 far away from the detection layer 20. For example, the base layer 31 is an ITO layer, and the hydrophobic layer 32 is an ITO layer doped with ZnO. The base layer 31 facilitates combining the hydrophobic layer 32 with the detection layer 20, thereby improving the stability of the detection chip. The contact angle of the surface of the hydrophobic layer 32 is relatively large, which facilitates the flow of the substance to be detected and the detection reagent into the detection hole 21.

For example, as illustrated in FIG. 2, the diameter of each detection hole 21 among the plurality of detection holes 21 is 2.0 microns-3.0 microns. For example, the cross-sectional shape, perpendicular to the base substrate 10, of the detection hole 21 is an inverted trapezoid; the length W2 of a long side of the inverted trapezoid (that is, the diameter of the detection hole 21 at the opening) is 2.0 microns-3.0 microns, such as 2.2 microns, 2.5 microns or 2.8 microns, etc.; the length W1 of a short side (that is, the diameter of the detection hole 21 on the bottom surface) is 1.0 micron-2.0 microns, such as 1.2 microns, 1.5 microns or 1.8 microns, etc.; and the distance W3 between adjacent detection holes 21 is 0.5 microns-1.5 microns, such as 0.8 microns, 1.0 micron or 1.2 microns, etc.

For example, as illustrated in FIG. 2, the depth H1 of each detection hole 21 among the plurality of detection holes 21 is 0.5 microns-1.5 microns, such as 0.8 microns, 1.0 micron or 1.2 microns, etc., and the thickness H2 of the microstructure 30 is 0.2 microns-0.8 microns, such as 0.4 microns, 0.5 microns or 0.6 microns, etc.

In the embodiments of the present disclosure, the depth or thickness of a structure refers to the dimension of the structure in a direction perpendicular to the base substrate 10, that is, the dimension in the vertical direction in FIG. 1B and FIG. 2.

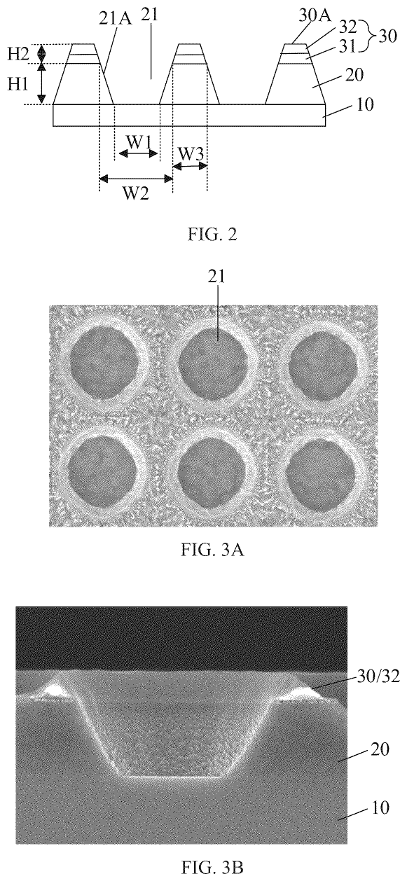

For example, FIG. 3A and FIG. 3B respectively illustrate scanning electron micrographs of a planar structure and a cross-sectional structure of the detection chip in the embodiment of FIG. 2. As illustrated in FIG. 3A, the cross-sectional shape, parallel to the base substrate 10, of the detection hole 21 is basically a circle. As illustrated in FIG. 3B, the base layer 31 and the hydrophobic layer 32 of the microstructure 30 have no clear boundary, and a slope is formed on the surface of the hydrophobic layer 32, so that the slope can be used to facilitate the entry of the substance to be detected and the detection reagent into the detection hole 21.

In the above-mentioned embodiments, a bottom layer of the detection hole 21 is the base substrate 10, such as a glass substrate, the detection layer 20 in the middle is a silicon oxide layer, and the ITO layer and the ITO layer doped with ZnO are provided on the silicon oxide layer. The silicon oxide layer is a hydrophilic material layer with a contact angle of about 2-5 degrees, which is close to a completely hydrophilic state, while the ITO layer doped with ZnO is a hydrophobic layer with a contact angle of about 100 degrees, and the hydrophobic layer completely covers the surface of the detection layer 20 between the plurality of detection holes 21. The structure has clear modification levels and boundaries inside and outside the detection hole, which can achieve the effect of hydrophobicity outside the detection hole and hydrophilicity at the edge of and inside of the detection hole. The structure is suitable for a reaction system in which the reaction solution is difficult to enter the detection hole, improves the efficiency of the reaction solution entering the detection hole and the efficiency of reaction process, thereby improving the quality and effect of detection.

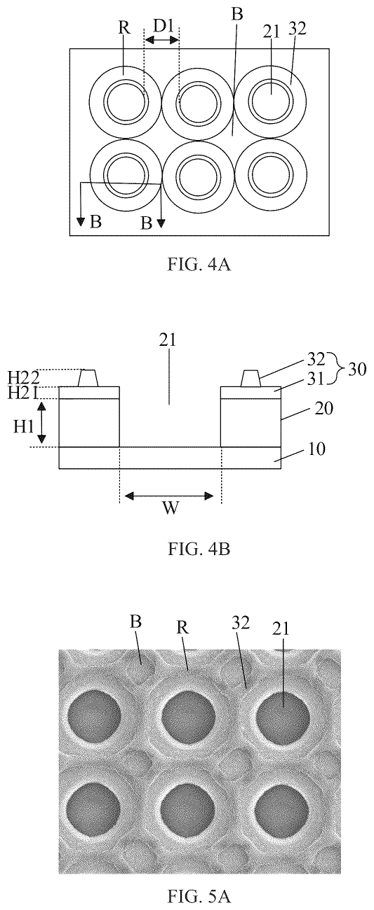

For example, FIG. 4A and FIG. 4B respectively illustrate a schematic plan view and a schematic cross-sectional view of another detection chip provided by at least one embodiment of the present disclosure, and FIG. 4B is obtained by taking along a line B-B in FIG. 4A. As illustrated in FIG. 4A and FIG. 4B, the surface of the detection layer 20 far away from the base substrate 10 is provided with a microstructure 30, and the microstructure 30 includes a base layer 31 and a hydrophobic layer 32 provided on a side of the base layer 31 far away from the detection layer 20. For example, the base layer 31 is a metal layer, and the hydrophobic layer 32 is a carbonized adhesive layer, that is, an adhesive layer that has been carbonized, such as a photoresist layer that has been carbonized.

For example, in some embodiments, the metal layer is an Al layer or a multilayer structure including an Al layer, such as a multilayer metal structure of Ti—Al—Ti.

For example, in some embodiments, as illustrated in FIG. 4A and FIG. 4B, the diameter W of each detection hole 21 among the plurality of detection holes 21 is 1.3 microns-2.5 microns, such as 1.5 microns, 2.0 microns or 2.2 microns, etc.; and the distance DI between adjacent detection holes 21 is 0.5 microns-2.0 microns, such as 0.8 microns, 1.0 micron, 1.5 microns or 1.8 microns, etc.

For example, as illustrated in FIG. 4B, the depth H1 of each detection hole 21 among the plurality of detection holes 21 is 0.5 microns-1.5 microns, such as 0.8 microns, 1.0 micron or 1.5 microns, etc., and the thickness H21+H22 of the microstructure 30 is 0.2 microns-1.0 micron, such as 0.5 microns, 0.8 microns or 1.0 micron, etc. For example, the thickness H21 of the base layer 31 in the microstructure 30 is 0.1 microns-0.2 microns, such as 0.11 microns, 0.13 microns or 0.15 microns, etc., and the thickness H22 of the hydrophobic layer 32 is 0.1 microns-0.8 microns, such as 0.3 microns, 0.5 microns or 0.6 microns, etc.

For example, FIG. 5A and FIG. 5B respectively illustrate scanning electron micrographs of a planar structure and a cross-sectional structure of the detection chip in the embodiment of FIG. 4A and FIG. 4B. As illustrated in FIG. 4A and FIG. 5A, the hydrophobic layer 32 includes a plurality of annular portions R respectively surrounding the plurality of detection holes 21, the plurality of annular portions R form a concave portion B between every four adjacent detection holes 21, and the material of the hydrophobic layer 32 does not exist in the concave portion B. As illustrated in FIG. 5B, the cross-sectional shape of each detection hole 21 perpendicular to the base substrate 10 is substantially rectangular, and the surface of the hydrophobic layer 32 presents an uneven topography, thereby facilitating the entry of the substance to be detected and the detection reagent formed thereon into the detection hole 21.

In the above-mentioned embodiment, the bottom layer of the detection hole 21 is a base substrate, such as a glass substrate, the detection layer 20 in the middle is a silicon oxide layer, and an Al layer and an etched and carbonized photoresist layer are provided above the silicon oxide layer. The silicon oxide layer is a hydrophilic material layer with a contact angle of about 2-5 degrees, which is close to a completely hydrophilic state, while the etched and carbonized photoresist layer is distributed around the surface of the detection hole 21 in a regular ring structure (e.g., with a diameter of about 1.3 microns-2.5 microns) to achieve a superhydrophobic effect with a contact angle of about 148 degrees.

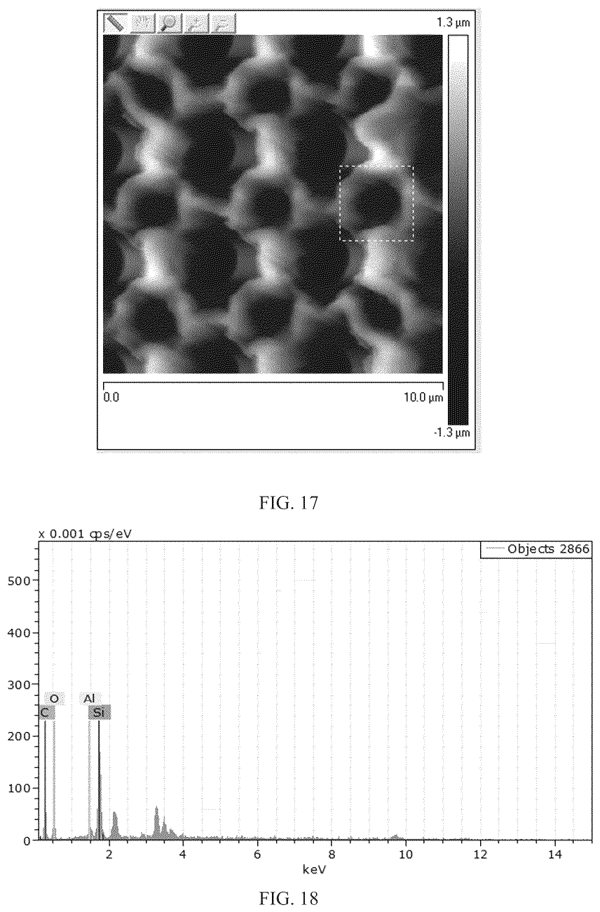

By AFM characterization, as illustrated in FIG. 17, the surface roughness Ra of the hydrophobic layer 32 formed on the surface of the detection layer 20 is 289 nm. By EDS clement energy spectrum analysis, as illustrated in FIG. 18, the results show that the main components of the hydrophobic layer 32 on the surface of the detection layer 20 are carbon, oxygen, silicon and aluminum, with the proportions of 63%, 25%, 11% and 1%, respectively. The structure achieves the effect of hydrophobicity outside the detection hole and hydrophilicity at the edge of and inside of the detection hole. The structure is suitable for a reaction system in which the reaction solution is difficult to enter the detection holes, improves the efficiency of the reaction solution entering the detection holes and the efficiency of the reaction process, thereby improving the quality and effect of detection.

For example, FIG. 6 illustrates a schematic cross-sectional view of another detection chip provided by at least one embodiment of the present disclosure. As illustrated in FIG. 6, in this embodiment, the detection chip further includes a base adhesive layer 40, and the base adhesive layer 40 is provided between the base substrate 10 and the detection layer 20 to improve the adhesion between the detection layer 20 and the base substrate 10.

For example, the base adhesive layer 40 includes a photoresist material, such as a negative photoresist material, mainly composed of olefinic substances, such as SOC-5004U adhesive material. The detection layer 20 includes a photoresist material, so that the detection layer 20 can be formed through simple exposure and development processes in the manufacturing process.

For example, as illustrated in FIG. 6, the detection chip further includes a first protective layer 51 and a second protective layer 52, the first protective layer 51 includes silicon dioxide and is provided between the base adhesive layer 40 and the detection layer 20, and the second protective layer 52 includes silicon dioxide and is provided on the side of the detection layer 20 far away from the base substrate 10. Because the photoresist material of the detection layer 20 is relatively unstable, the structural stability of the detection layer 20 can be improved by providing protective layers including silicon dioxide on the upper and lower sides of the detection layer 20, respectively.

For example, as illustrated in FIG. 6, the cross-sectional shape of at least one detection hole 21 (for example, each detection hole 21) among the plurality of detection holes 21 perpendicular to the base substrate 10 includes two arc-shaped edges RC protruding toward each other, that is, the cross-section of the inner wall of each detection hole 21 is arc-shaped, and the arc-shaped inner wall can guide the substance to be detected and the detection reagent formed thereon to enter the detection hole 21.

For example, in some embodiments, the diameter of each detection hole 21 among the plurality of detection holes 21 is 0.2 microns-2.2 microns. For example, because the inner wall of each detection hole 21 is arc-shaped, the diameter of each detection hole 21 is different at different positions.

For example, as illustrated in FIG. 6, a first cross-section of the detection hole 21 parallel to the base substrate 10 has a first diameter W1, a second cross-section of the detection hole 21 parallel to the base substrate 10 has a second diameter W2, and a third cross-section of the detection hole 21 parallel to the base substrate 10 has a third diameter W3; the second cross-section is located on a side of the first cross-section far away from the base substrate 10, and the third cross-section is located on a side of the second cross-section far away from the base substrate 10; and the second diameter W2 is smaller than the first diameter W1 and the third diameter W3, that is, the diameter of the detection hole 21 is smaller in the middle and larger at the upper and lower ends.

For example, in some embodiments, as illustrated in FIG. 6, the diameter W1 of each detection hole 21 at the bottom is 0.4 microns-1.8 microns, such as 0.6 microns, 0.8 microns, 1.0 micron, 1.3 microns or 1.5 microns, etc.; the diameter W2 of each detection hole 21 at the middle position is 0.3 microns-1.5 microns, such as 0.5 microns, 0.7 microns, 0.9 microns, 1.2 microns or 1.3 microns, etc.; and the diameter W3 of each detection hole 21 at the opening is 0.6 microns-2.2 microns, such as 0.8 microns, 1.0 micron, 1.3 microns, 1.5 microns, 1.8 microns or 2.0 microns, etc. For example, the depth H1 of each detection hole 21 is 0.5 microns-1.5 microns, such as 0.8 microns, 1.0 micron or 1.2 microns, etc.

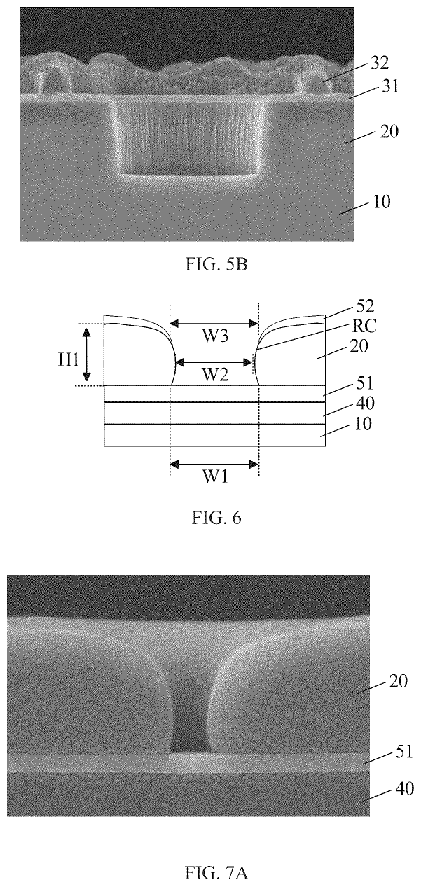

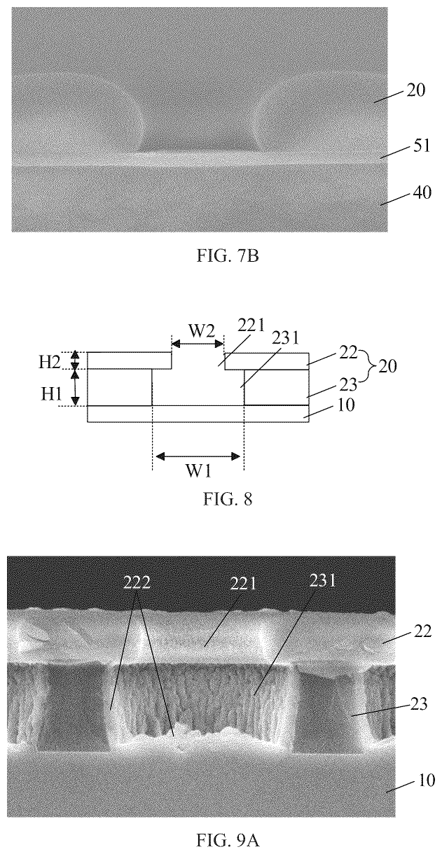

For example, FIG. 7A and FIG. 7B respectively illustrate scanning electron micrographs of two cross-sectional structures of the detection chip in the embodiment of FIG. 6, and the diameters of the detection holes 21 are different in the two cross-sectional structures.

For example, in the embodiment of FIG. 7A, the diameter W1 of each detection hole 21 at the bottom is about 0.4 microns, the diameter W2 of each detection hole 21 at the middle position is about 0.3 microns, the diameter W3 of each detection hole 21 at the opening is about 0.6 microns, and the depth H1 of each detection hole 21 is about 1.1 microns.

For example, in the embodiment of FIG. 7B, the diameter W1 of each detection hole 21 at the bottom is about 1.7 microns, the diameter W2 of each detection hole 21 at the middle position is about 1.4 microns, the diameter W3 of each detection hole 21 at the opening is about 2.1 microns, and the depth Hl of each detection hole 21 is about 1.1 microns.

In the above-mentioned embodiment, the overall shape of each detection hole 21 is similar to a dumbbell shape, and the structure is open at the opening and has a smooth and fluent surface, which is very beneficial for the reaction solution that is difficult to enter the detection hole 21 to enter the detection hole 21. The middle of the detection hole 21 shrinks and becomes smaller, which helps to reduce liquid overflow caused by heating during the reaction process. Therefore, the detection hole 21 of this shape not only effectively increases the efficiency of the reaction solution entering the detection hole 21, but also reduces the possibility of reaction solution crossing holes during the reaction process, and is more suitable for the reaction solution with poor fluidity, thereby improving the quality and effect of the detection.

For example, FIG. 8 illustrates a schematic cross-sectional view of another detection chip provided by at least one embodiment of the present disclosure. As illustrated in FIG. 8, in this embodiment, the detection layer 20 includes a first sub-detection layer 23 and a second sub-detection layer 22 provided on a side of the first sub-detection layer 23 far away from the base substrate 10, and the first sub-detection layer 23 is an adhesive layer, for example, includes a photoresist material, such as a negative photoresist material, mainly composed of olefinic substances, such as SOC-5004U adhesive material. The second sub-detection layer 22 is a silicon oxide layer, and the detection hole 21 includes a first sub-detection hole 231 provided in the first sub-detection layer 23 and a second sub-detection hole 221 provided in the second sub-detection layer 22 that are interpenetrated with each other.

For example, as illustrated in FIG. 8, the orthographic projection of the second sub-detection hole 221 on the base substrate 10 is located inside the orthographic projection of the first sub-detection hole 231 on the base substrate 10, thereby forming the detection hole 21 with a smaller opening and a larger cavity.

For example, in some embodiments, the diameter W1 of the first sub-detection hole 231 is 1.0 micron-2.5 microns, such as 1.5 microns, 1.8 microns, 2.0 microns or 2.2 microns, etc., and the depth H1 of the first sub-detection hole 231 is 1.0 micron-1.8 microns, such as 1.2 microns, 1.5 microns or 1.8 microns, etc.; and the diameter W2 of the second sub-detection hole 221 is 0.6 microns-1.8 microns, such as 0.8 microns, 1.0 micron, 1.2 microns, 1.4 microns or 1.6 microns, etc., and the depth H2 of the second sub-detection hole 221 is 0.4 microns-0.8 microns, such as 0.5 microns, 0.6 microns or 0.7 microns, etc.

For example, FIG. 9A and FIG. 9B respectively illustrate scanning electron micrographs of a cross-sectional structure and a three-dimensional structure of the detection chip in the embodiment of FIG. 8. As illustrated in FIG. 9A and FIG. 9B, the bottom and side wall of the first sub-detection hole 231 are also formed with the material 222 of the second sub-detection layer 22, so that the opening edge of the first sub-detection hole 231 is covered by the second sub-detection layer 22. In this embodiment, the diameter W1 of the first sub-detection hole 231 is about 2.2 microns, and the depth H1 of the first sub-detection hole 231 is about 1.2 microns; and the diameter W2 of the second sub-detection hole 221 is about 1.4 microns, and the depth H2 of the second sub-detection hole 221 is about 0.6 microns. The thickness of the material 222 of the second sub-detection layer 22 formed on the bottom and the side wall of the first sub-detection hole 231 is about 0.2 microns.

In the above-mentioned embodiment, the overall shape of the detection hole 21 is similar to a shape of a vase, and the structure has a smooth slope at the opening, which is beneficial for the reaction solution to enter the detection hole 21; moreover, the diameter of the opening is small, thereby well reducing the risk of liquid overflow during heating in the reaction process. Therefore, the structure not only effectively increases the efficiency of the reaction solution entering the detection hole 21, but also reduces the possibility of reaction solution crossing holes during the reaction process, and is more suitable for a detection process with a more intense reaction process, thereby improving the quality and effect of the detection.

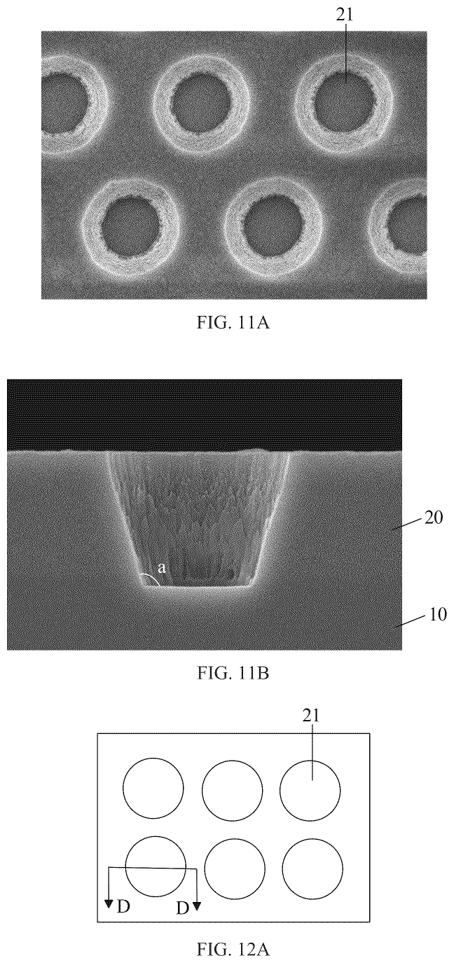

For example, FIG. 10A and FIG. 10B respectively illustrate a schematic plan view and a schematic cross-sectional view of another detection chip provided by at least one embodiment of the present disclosure, and FIG. 10B is obtained by taking along a line C-C in FIG. 10A. As illustrated in FIG. 10A and FIG. 10B, a cross-sectional shape, parallel to the base substrate 10, of each of at least part of detection holes 21 (for example, all the detection holes 21) among the plurality of detection holes 21 is a circle, and a cross-sectional shape, perpendicular to the base substrate 10, of each of at least part of detection holes 21 among the plurality of detection holes 21 is an inverted trapezoid. For example, as illustrated in FIG. 10A, the plurality of detection holes 21 are arranged in a staggered manner so that more detection holes 21 can be arranged in the same area to increase the ratio of holes.

For example, in some embodiments, as illustrated in FIG. 10B, the length W2 of the long side of the inverted trapezoid (that is, the diameter of the detection hole 21 at the opening) is 1.2 microns-2.2 microns, such as 1.5 microns, 1.8 microns, 2.0 microns or 2.2microns, etc., the length W1 of the short side of the inverted trapezoid (that is, the diameter of the detection hole 21 on the bottom surface) is 0.5 microns-1.8 microns, such as 0.8 microns, 1.0 micron, 1.2 microns, 1.5 microns or 1.7 microns, etc., and the height H1 of the inverted trapezoid is 1.0 micron-1.8 microns, such as 1.2 microns, 1.5 microns or 1.7 microns, etc.

For example, FIG. 11A and FIG. 11B respectively illustrate scanning electron micrographs of a planar structure and a cross-sectional structure of the detection chip in the embodiment of FIG. 10A and FIG. 10B. As illustrated in FIG. 11A, the cross-sectional shape of each detection hole 21 parallel to the base substrate 10 is basically a circle, and as illustrated in FIG. 11B, the cross-sectional shape of each detection hole 21 perpendicular to the base substrate 10 is basically an inverted trapezoid. However, due to the process and other reasons, the cross-section of the side wall of each detection hole 21 is slightly curved and not strictly straight, and the side wall of each detection hole 21 is in the shape of fish scales or burrs. For example, as illustrated in FIG. 11B, the side wall of each detection hole 21 forms an included angle a with the bottom surface, and the included angle a ranges from about 70 degrees to 85 degrees, such as 75 degrees, 80 degrees, or 85 degrees, etc.

Thus, in the above-mentioned detection hole 21 whose overall shape is an inverted trapezoid, because the diameter of the detection hole 21 is relatively large at the opening, which is extremely beneficial for the reaction solution to enter the detection hole 21 and the discharge of gas in the detection hole 21, thereby reducing the influence of residual air bubbles in the detection hole 21. However, this structure is easy to cause the liquid in the reaction process to overflow, so this structure is more suitable for the detection situation where the reaction is stable but the fluidity of the reaction solution is poor and the reaction solution is difficult to enter the detection hole 21. That is, this structure effectively increases the efficiency of the reaction solution entering the detection hole 21 and the discharge of air bubbles, but the stability of the liquid is limited during the reaction process.

For example, FIG. 12A and FIG. 12B respectively illustrate a schematic plan view and a schematic cross-sectional view of another detection chip provided by at least one embodiment of the present disclosure, and FIG. 12B is obtained by taking along a line D-D in FIG. 12A. As illustrated in FIG. 12A and FIG. 12B, a cross-sectional shape, parallel to the base substrate 10, of each of at least part of detection holes 21 among the plurality of detection holes 21 is a circle, and a cross-sectional shape, perpendicular to the base substrate 10, of each of at least part of detection holes 21 among the plurality of detection holes 21 is a rectangle.

For example, the diameter of each of at least part of detection holes 21 among the plurality of detection holes 21 is 1.0 micron-2.2 microns, such as 1.2 microns, 1.5 microns, 1.8 microns or 2.0 microns, etc., and the depth H of each of at least part of detection holes 21 among the plurality of detection holes 21 is 1.0 micron-1.8 microns, such as 1.2 microns, 1.5 microns or 1.8 microns, etc.

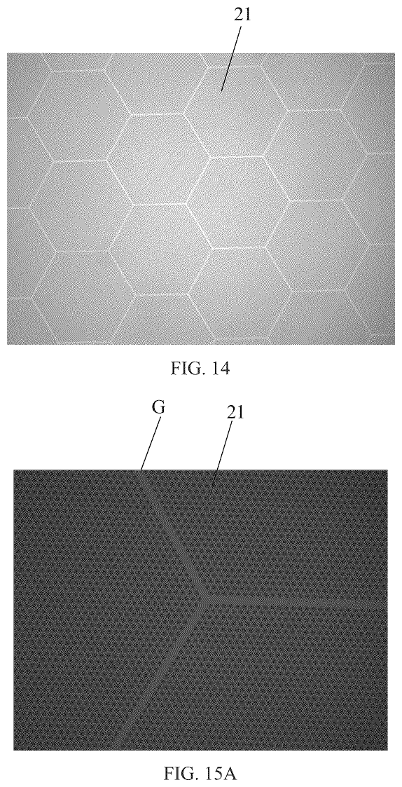

For example, FIG. 13A and FIG. 13B respectively illustrate the scanning electron micrographs of a planar structure and a cross-sectional structure of the detection chip in the embodiment of FIG. 12A and FIG. 12B. As illustrated in FIG. 13A, the cross-sectional shape of each detection hole 21 parallel to the base substrate 10 is basically a circle, and as illustrated in FIG. 13B, the cross-sectional shape of each detection hole 21 perpendicular to the base substrate 10 is basically a rectangle. However, due to the process and other reasons, the cross-section of the side wall of each detection hole 21 is slightly curved and not strictly straight, and the side wall of each detection hole 21 is in the shape of fish scales. For example, a tangent is made at each position of the micro-arc of the cross-section of the side wall of each detection hole 21, the tangent forms an angle b with the bottom surface, and the angle b ranges from about 75 degrees to 90 degrees, such as 79-86 degrees, such as 80 degrees, 83 degrees or 85 degrees, etc.

Therefore, in the above-mentioned embodiment, the overall shape of each detection hole 21 is a relatively standard cylinder, the side wall of this structure is almost vertical, the efficiency of the reaction solution entering the detection hole 21 and the possibility of liquid crossing the holes during the reaction are moderate, and it is suitable for the detection process that requires both the liquid entering the detection hole 21 and the stability of the reaction.

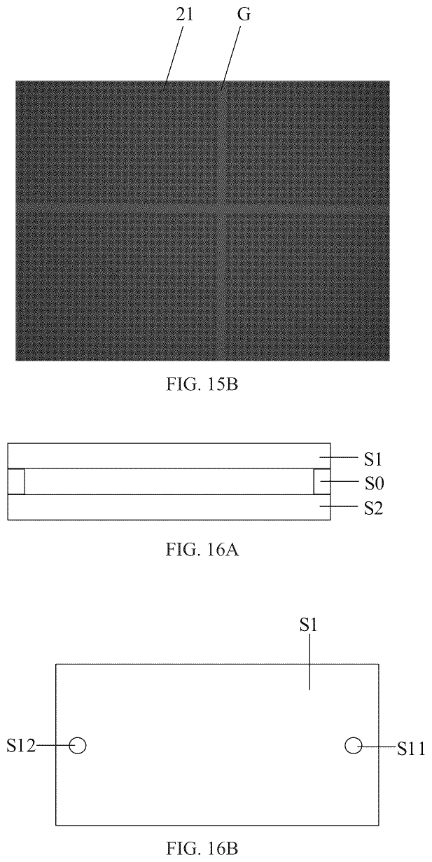

For example, the above-mentioned embodiments are introduced by taking the cross-sectional shape of each detection hole 21 parallel to the base substrate 10 as a circle as an example. In other embodiments, as illustrated in FIG. 14, the cross-sectional shape of each detection hole 21 parallel to the base substrate 10 may also be a hexagon, or a polygon such as a square or a pentagon in other embodiments. For example, as illustrated in FIG. 15A and FIG. 15B, the plurality of detection holes 21 are divided into a plurality of groups, and the plurality of groups of detection holes 21 are separated by an interval G. The planar shape of each group of detection holes 21 may be formed into a hexagon, as illustrated in FIG. 15A, or formed into a rectangle, as illustrated in FIG. 15B, or formed into other figures such as a pentagon in other embodiments.

For example, FIG. 14 and FIG. 15A respectively illustrate layout diagrams of a hexagonal detection hole array under a microscope at 10 times and 100 times. As illustrated in FIG. 14, the cross-sectional shape of the detection hole parallel to the base substrate 10 is a hexagon, such as an equilateral hexagon; as illustrated in FIG. 15A, a plurality of detection holes form a group, each group of detection holes is roughly hexagonal, the interval G between adjacent groups of detection holes is a size of 1-3 columns of detection holes, and the side length of each group of detection holes (that is, the side length of the hexagon presented by each group of detection holes) accommodates 50-70 detection holes. The diameter of each detection hole is about 0.5 microns-2.5 microns, the distance between adjacent detection holes is about 0.5 microns-2.5 microns, and the depth of the detection hole is about 0.5 microns-2.5 microns. Utilizing the above arrangement and distance of the detection holes can optimize the ratio of the detection holes, achieve the accurate positioning and identification, increase the reaction volume and identification speed, and provide a good foundation for the detection.

For example, in some embodiments, the detection chip further includes a cover plate. For example, FIG. 16A illustrates a schematic cross-sectional view of another detection chip provided by at least one embodiment of the present disclosure, and FIG. 16B illustrates a schematic plan view of a cover plate. As illustrated in FIG. 16A, the detection chip S2 is bonded with a cover plate S1 through a sealant S0, as illustrated in FIG. 16B, the cover plate S1 includes a sample inlet S11 and a sample outlet S12, the sample inlet S11 is configured to introduce liquids such as substance to be detected or detection reagent, and the sample outlet S12 is configured to exhaust or discharge the remaining substance to be detected or detection reagent after the detection is completed. For example, the sample inlet S11 and the sample outlet S12 are respectively provided on opposite sides of the cover plate S1. For example, the cover plate S1 is a transparent cover plate such as a glass cover plate or an acrylic cover plate, etc.

For example, the planar shape of each of the sample inlet S11 and the sample outlet S12 is a regular shape such as a circle (as illustrated in the figure), an ellipse or a square, so as to facilitate operations such as adding samples or exhausting remaining samples quickly and efficiently. For example, in some embodiments, the sample inlet S11 and the sample outlet S12 may each have a guide structure to facilitate the inflow or outflow of the substance to be detected or the detection reagent.

For example, in some embodiments, the overall dimension of the detection chip is about 25 mm*65 mm, and there are about 3.5×10⁸ detection holes 21 within this dimension range, the detection chip has a relatively high detection hole ratio and has a high throughput.

At least one embodiment of the present disclosure further provides a manufacturing method of a detection chip, including providing a base substrate and forming a detection layer on the base substrate; the detection layer includes a plurality of detection holes, an inner wall of each of at least part of detection holes among the plurality of detection holes is hydrophilic, and a contact angle of the inner wall is within 30 degrees.

For example, in some embodiments, the surface of the detection layer far away from the base substrate is hydrophobic relative to the inner wall of the at least part of detection holes, or the surface of the detection layer far away from the base substrate is provided with a microstructure. In this case, the manufacturing method further includes: performing hydrophobic treatment on the surface of the detection layer far away from the base substrate to enable the contact angle of the surface to be 80-150 degrees; or forming a microstructure on the surface of the detection layer far away from the base substrate, and the contact angle of at least the surface of the microstructure far away from the base substrate is 80-150 degrees.

For example, the hydrophobic treatment on the surface of the detection layer away from the base substrate is chemical modification, such as connecting some hydrophobic functional groups to the above-mentioned surface, etc.

For example, for the detection chip in the embodiment of FIG. 2, the manufacturing method includes the following steps.

First, referring to FIG. 2, a base substrate is provided, and then a detection material layer is formed on the base substrate. A base material layer is formed on a side of the detection material layer far away from the base substrate, a hydrophobic material layer is formed on a side of the base material layer far away from the base substrate, and a photoresist pattern is formed on a side of the hydrophobic material layer far away from the base substrate. Then the hydrophobic material layer, the base material layer and the detection material layer are patterned by using the photoresist pattern as a mask to form the detection layer and the microstructure. Thus, the detection layer and the microstructure are simultaneously formed through one patterning process.

For example, in a manufacturing process, the base substrate is firstly cleaned, and then the detection material layer, such as a silicon oxide material layer, is formed on the base substrate by plasma-enhanced chemical vapor deposition (PECVD). During the process of PECVD, the RF power used is about 650 W, the ion deposition distance is about 710 mil (1 mil-0.0254 mm), the chamber pressure is about 1500 mTorr (1 mTorr=0.133 Pa), the gases are SiH₄, N₂ and N₂O, and the flow rates of SiH₄, N₂ and N₂O are 85 sccm, 500 sccm, and 1850 sccm, respectively, in this case, the deposition thickness of the silicon oxide material layer is about 1000 nanometers; then, the base material layer, such as an ITO layer, is formed on the silicon oxide material layer by a sputtering process, during the process of the sputtering process, the power applied to the target material is about 10 KW, and the chamber pressure is about 0.4 Pa, and in this case, the sputtering thickness of the ITO layer is about 300 nanometers; after that, the hydrophobic material layer, such as an ITO layer doped with ZnO, is formed on the base material layer by a sputtering process, during the process of the sputtering process, the power applied to the target material is about 10 KW, and the chamber pressure is about 0.4 Pa, and in this case, the sputtering thickness of the ITO layer doped with ZnO is about 100 nanometers.

Afterwards, a photoresist material layer is formed on a side of the hydrophobic material layer far away from the base substrate by a process such as coating, and then the photoresist material layer is exposed and developed to form the photoresist pattern. For example, the thickness of the photoresist material layer is about 1.5 microns, the exposure intensity is about 100 mJ, and the development time is about 60 s. The photoresist pattern is post-baked after development, for example, the post-baking temperature is about 130° C. and the time is about 2 minutes.

After the photoresist pattern is formed, the photoresist pattern is used as a mask, and a dry etching process, such as ICP dry etching, is used to etch the hydrophobic material layer, the base material layer and the detection material layer. During the etching process, the chamber pressure is about 150 mTorr, the RF power is about 800 W, and in this case, the flow rate of oxygen is 400 sccm, and the time is 10s; after that, the chamber pressure is adjusted to about 100 mTorr, the RF power is about 1000 W, Cl₂ and Ar₂ are introduced, the flow rates of Cl₂ and Ar₂ are 100 sccm and 80 sccm respectively, and the time is 320 s; after that, the chamber pressure is adjusted to about 130 mTorr, the RF power is about 800 w, oxygen and CF₄ are introduced, the flow rates of oxygen and CF₄ are 400 sccm and 80 sccm respectively, and the time is 300 s. In this way, the detection layer 21, the base layer 31 and the hydrophobic layer 32 as illustrated in FIG. 2 are formed by etching.

After the etching is completed, the remaining photoresist above the hydrophobic layer 32 is cleaned, and then the obtained structure is cut, for example, to form at least one detection chip whose dimension and shape meet the requirements, as illustrated in FIG. 2.

For example, for the detection substrate in the embodiment illustrated in FIG. 4A and FIG. 4B, the manufacturing process includes the following steps.

First, a base substrate is provided, and then a detection material layer is formed on the base substrate. A base material layer is formed on a side of the detection material layer far away from the base substrate, a photoresist pattern is formed on a side of the base material layer far away from the base substrate, the base material layer and the detection material layer are patterned with the photoresist pattern as a mask to form the detection layer and the base layer, and then the photoresist pattern is carbonized to form a microstructure.

For example, in a manufacturing process, the base substrate is firstly cleaned, and then the detection material layer, such as a silicon oxide material layer, is formed on the base substrate by using plasma-enhanced chemical vapor deposition (PECVD). During the process of PECVD, the RF power used is about 650 W, the ion deposition distance is about 710 mil (1 mil=0.0254 mm), the chamber pressure is about 1500 mTorr (1 mTorr=0.133 Pa), the gases are SiH₄, N₂ and N₂O, and the flow rates of SiH₄, N₂ and N₂O are 85 sccm, 500 sccm, and 1850 sccm, respectively, in this case, the deposition thickness of the silicon oxide material layer is about 1000 nanometers; then, the base material layer, for example, a metal material layer, such as an Al layer, is formed on the silicon oxide material layer by a sputtering process, during the process of the sputtering process, the power applied to the target material is about 10 KW, and the chamber pressure is about 0.4 Pa, and in this case, the sputtering thickness of the ITO layer is about 100 nanometers.

Afterwards, a photoresist material layer is formed on a side of the Al layer far away from the base substrate by a process such as coating, and then the photoresist material layer is exposed and developed to form the photoresist pattern. For example, the formation thickness of the photoresist material layer is about 1.5 microns, the exposure intensity is about 100 mJ, and the development time is about 60 s. The photoresist pattern is post-baked after development, for example, the post-baking temperature is about 130° C. and the time is about 2 minutes.

After the photoresist pattern is formed, the photoresist pattern is used as a mask, and a dry etching process, such as ICP dry etching, is used to etch the base material layer and the detection material layer. During the etching process, the chamber pressure is about 150 mTorr, the RF power is about 800 W, and in this case, the flow rate of oxygen is 400 sccm, and the time is 10 s; after that, the chamber pressure is adjusted to about 100 mTorr, the RF power is about 1000 W, Cl₂ and Ar₂ are introduced, the flow rates of Cl₂ and Ar₂ are 40 sccm and 100 sccm respectively, and the time is 150 s; after that, the chamber pressure is adjusted to about 130 mTorr, the RF power is about 800 w, oxygen and CF₄ are introduced, the flow rates of oxygen and CF₄ are 400 sccm and 80 sccm respectively, and the time is 300 s. In this way, the detection layer 21 and the base layer 31 as illustrated in FIG. 2 are formed by etching.

Afterwards, the photoresist pattern is carbonized, for example, at a temperature of about 150° C.-180° C., to form the hydrophobic layer 32.

Finally, the obtained structure is cut, for example, to form at least one detection chip whose dimension and shape meet the requirements, as illustrated in FIG. 4A and FIG. 4B.

For example, for the detection chip in the embodiment illustrated in FIG. 6, in the manufacturing process, first, the base substrate is cleaned, and then a coating process is used to form an adhesive layer on the base substrate. For example, a spin coater is used in the coating process, the rotation speed of the spin coater is about 700 rpm, the time is 10 s, and the pressure is 28; then the coated adhesive layer is pre-baked at about 80° C. twice, the first pre-baking time is 40 s, and the second pre-baking time is 80 s, and then the adhesive layer is exposed, and then the adhesive layer is post-baked at about 230° C. for about 1 hour. In this case, the thickness of the adhesive layer formed is about 1.5 microns, thereby forming the adhesive layer 40.

Then, a first protective material layer, such as a silicon oxide material layer, is formed on the adhesive layer by using PECVD. During the PECVD process, the RF power used is about 900 W, the ion deposition distance is about 850 mil (1 mil=0.0254 mm), the chamber pressure is about 1000 mTorr (1 mTorr=0.133 Pa), the gases are SiH₄, N₂ and N₂O, and the flow rates of SiH₄, N₂ and N₂O are about 85 sccm, 500 sccm and 1850 sccm respectively. In this case, the deposition thickness of the silicon oxide material layer is about 100 nanometers, thereby forming a first protective layer 51. Then, a detection material layer, such as a photoresist material layer, is formed on the first protective material layer. For example, the photoresist material layer is formed by using a coating process, the thickness of the photoresist material layer is about 1.0 micron-3.0 microns, and the photoresist material layer is exposed, developed and post-baked, in which the intensity of the exposure is about 65 mJ-120 mJ, the development time is about 50 s-60 s, the temperature of the post-baking is about 130° C., and the time is about 2 minutes, thereby forming the detection layer 21.

Then, a second protective material layer, such as a silicon oxide material layer, is formed by using PECVD. During the PECVD process, the RF power used is about 1200 W, the ion deposition distance is about 950 mil (1 mil-0.0254 mm), the chamber pressure is about 900 mTorr (1 mTorr-0.133 Pa), the gases are SiH₄, N₂ and N₂O, and the flow rates of SiH₄, N₂ and N₂O are about 85 sccm, 500 sccm and 1850 sccm respectively. In this case, the deposition thickness of the silicon oxide material layer is about 10 nanometers, thereby forming a second protective layer 52.

Finally, the obtained structure is cut, for example, to form at least one detection chip whose dimension and shape meet the requirements, as illustrated in FIG. 6.

For example, for the detection chip in the embodiment illustrated in FIG. 8, in the manufacturing process, first, the base substrate is cleaned, and then a coating process is used to form an adhesive layer on the base substrate. For example, a spin coater is used in the coating process, the rotation speed of the spin coater is about 700 rpm, the time is 10 s, and the pressure is 28; then the coated adhesive layer is pre-baked at about 80° C. twice, the first pre-baking time is 40 s, and the second pre-baking time is 80 s, and then the adhesive layer is exposed, and then the adhesive layer is post-baked at about 230° C. for about 1 hour. In this case, the thickness of the adhesive layer formed is about 1.0 micron-1.5 microns, thereby forming the first detection material layer.

Then, a second detection material layer, such as a silicon oxide material layer, is formed on the adhesive layer by using PECVD. During the PECVD process, the RF power used is about 700 W, the ion deposition distance is about 600 mil (1 mil=0.0254 mm), the chamber pressure is about 1000 mTorr (1 mTorr=0.133 Pa), the gases are SiH₄, N₂ and N₂O, and the flow rates of SiH₄, N₂ and N₂O are about 85 sccm, 500 sccm and 1850 sccm respectively. In this case, the deposition thickness of the silicon oxide material layer is about 100 nanometers. Then, a photoresist material layer is formed on the second detection material layer, for example, a photoresist material layer is formed by a coating process, the thickness of the photoresist material layer is about 1.5 microns, and the photoresist material layer is exposed, developed and post-baked, in which the intensity of the exposure is about 85 mJ, the development time is about 50 s, the temperature of the post-baking is about 130° C., and the time is about 2 minutes, thereby forming a photoresist pattern.

Then, the first detection material layer and the second detection material layer are etched using the photoresist pattern as a mask and using a dry etching process, such as ICP dry etching. During the etching process, the chamber pressure is about 150 mTorr, the RF power is about 800 W, and in this case, the flow rate of oxygen is 400 sccm, and the time is 10 s; after that, the chamber pressure is adjusted to about 100 mTorr, the RF power is about 600 W, CF₄ and oxygen are introduced, the flow rates of CF₄ and oxygen are 100 sccm and 80 sccm respectively, and the time is 320 s; after that, the chamber pressure is adjusted to about 130 mTorr, the RF power is about 800 w, oxygen and CF₄ are introduced, the flow rates of oxygen and CF₄ are 400 sccm and 80 sccm respectively, and the time is 60 s; after that, the chamber pressure is adjusted to about 60 mTorr, the RF power is about 800 w, oxygen and CF₄ are introduced, the flow rates of oxygen and CF₄ are 100 sccm and 200 sccm respectively, and the time is 250 s. In this way, the first sub-detection layer 23 and part of the second sub-detection layer 22 as illustrated in FIG. 8 are formed by etching. After the etching is completed, the remaining photoresist pattern is cleaned.

Then, a third detection material layer, such as a silicon oxide material layer, is formed by using PECVD. During the PECVD process, the RF power used is about 1200 W, the ion deposition distance is about 950 mil (1 mil-0.0254 mm), the chamber pressure is about 900 mTorr (1 mTorr=0.133 Pa), the gases are SiH₄, N₂ and N₂O, and the flow rates of SiH₄, N₂ and N₂O are about 85 sccm, 500 sccm and 1850 sccm respectively. In this case, the deposition thickness of the silicon oxide material layer is about 300 nanometers, thereby forming another part of the second sub-detection layer 22, that is, the second sub-detection layer 22 is formed by the above-mentioned second detection material layer and the third detection material layer together.

Finally, the obtained structure is cut, for example, to form at least one detection chip whose dimension and shape meet the requirements, as illustrated in FIG. 8.

For example, for the detection chip in the embodiment illustrated in FIG. 10A and FIG. 10B, in the manufacturing process, the base substrate is firstly cleaned, and then the detection material layer, such as a silicon oxide material layer, is formed on the base substrate by PECVD. During the process of PECVD, the RF power used is about 650 W, the ion deposition distance is about 710 mil (1 mil=0.0254 mm), the chamber pressure is about 1500 mTorr (1 mTorr=0.133 Pa), the gases are SiH₄, N₂ and N₂O, and the flow rates of SiH₄, N₂ and N₂O are 85 sccm, 500 sccm, and 1850 sccm, respectively, in this case, the deposition thickness of the silicon oxide material layer is about 1000 nanometers; then, an auxiliary material layer, for example, a metal material layer, such as an Al layer, is formed on the detection material layer by a sputtering process, during the process of the sputtering process, the power applied to the target material is about 10 KW, and the chamber pressure is about 0.4 Pa, and in this case, the sputtering thickness of the Al layer is about 200 nanometers.

Afterwards, a photoresist material layer is formed on the Al layer, for example, a photoresist material layer is formed by a coating process with a thickness of about 1.5 microns, and the photoresist material layer is exposed, developed and post-baked, in which the exposure intensity is about 100 mJ, the development time is about 50 s, the post-baking temperature is about 130° C., and the time is about 2 minutes, thereby forming a photoresist pattern.

Then, the auxiliary material layer and the detection material layer are etched using the photoresist pattern as a mask and using a dry etching process, such as ICP dry etching. During the etching process, the chamber pressure is about 150 mTorr, the RF power is about 800 W, and in this case, the flow rate of oxygen is 400 sccm, and the time is 10 s; after that, the chamber pressure is adjusted to about 100 mTorr, the RF power is about 1000 W, Cl₂ and Ar₂ are introduced, the flow rates of Cl₂ and Ar₂ are 100 sccm and 80 sccm respectively, and the time is 320 s; after that, the chamber pressure is adjusted to about 130 mTorr, the RF power is about 800 w, oxygen and CF₄ are introduced, the flow rates of oxygen and CF₄ are 400 sccm and 80 sccm respectively, and the time is 60 s; after that, the chamber pressure is adjusted to about 60 mTorr, the RF power is about 800 w, Cl₂ and Ar₂are introduced, the flow rates of Cl₂ and Ar₂ are 200 sccm and 100 sccm respectively, and the time is 250 s.

Afterwards, the residual photoresist is cleaned, and the residual Al material is etched by a wet etching process. For example, in the wet etching process, the temperature is about 41° C., the etching is performed three times at intervals, and each time is about 25 seconds, thereby forming the detection layer 20 as illustrated in FIG. 10A and FIG. 10B.

Finally, the obtained structure is cut, for example, to form at least one detection chip whose dimension and shape meet the requirements, as illustrated in FIG. 10A and FIG. 10B.

For example, for the detection chip in the embodiment illustrated in FIG. 12A and FIG. 12B, in the manufacturing process, the base substrate is firstly cleaned, and then the detection material layer, such as a silicon oxide material layer, is formed on the base substrate by PECVD. During the process of PECVD, the RF power used is about 1000 W, the ion deposition distance is about 800 mil (1 mil-0.0254 mm), the chamber pressure is about 2000 mTorr (1 mTorr=0.133 Pa), the gases are SiH₄, N₂ and N₂O, and the flow rates of SiH₄, N₂ and N₂O are 100 sccm, 500 sccm, and 1950 sccm, respectively, in this case, the deposition thickness of the silicon oxide material layer is about 1000 nanometers; then, an auxiliary material layer, for example, an ITO layer, is formed on the detection material layer by a sputtering process, during the process of the sputtering process, the power applied to the target material is about 10 KW, and the chamber pressure is about 0.4 Pa, and in this case, the sputtering thickness of the ITO layer is about 200 nanometers.

Afterwards, a photoresist material layer is formed on the ITO layer, for example, a photoresist material layer is formed by a coating process with a thickness of about 1.5 microns, and the photoresist material layer is exposed, developed and post-baked, in which the exposure intensity is about 100 mJ, the development time is about 60 s, the post-baking temperature is about 130° C., and the time is about 2 minutes, thereby forming a photoresist pattern.

Then, the auxiliary material layer and the detection material layer are etched using the photoresist pattern as a mask and using a dry etching process, such as ICP dry etching.

During the etching process, the chamber pressure is about 150 mTorr, the RF power is about 800 W, and in this case, the flow rate of oxygen is 400 sccm, and the time is 10 s; after that, the chamber pressure is adjusted to about 100 mTorr, the RF power is about 1000 W, Cl₂ and Ar₂ are introduced, the flow rates of Cl₂ and Ar₂ are 100 sccm and 80 sccm respectively, and the time is 320 s; after that, the chamber pressure is adjusted to about 80 mTorr, the RF power is about 1000 w, oxygen and CHF₃ are introduced, the flow rates of oxygen and CHF₃ are 80 sccm and 200 sccm respectively, and the time is 300 s.

Afterwards, the residual photoresist is cleaned, and the residual ITO material is etched by a wet etching process. For example, in the wet etching process, the temperature is about 42° C., the etching is performed three times at intervals, and each time is about 30 seconds, thereby forming the detection layer 20 as illustrated in FIG. 12A and FIG. 12B.

At least one embodiment of the present disclosure further provides a detection method using the above-mentioned detection chip, the detection method includes: preparing a reaction wash solution; preparing a reaction mother solution; preparing a plurality of groups of reaction solution by using the reaction mother solution, in which the plurality of groups of reaction solution includes a first group of reaction solution; introducing the reaction wash solution into the detection chip, lowering the temperature of the detection chip to 3° C.-6° C., for example, 4° C. or 5° C., etc., and taking a fluorescence image; introducing the first group of reaction solution into the detection chip, raising the temperature of the detection chip to 60° C.-70° C., for example, 65° C., etc., and taking a fluorescence image.

For example, in some embodiments, the plurality of groups of reaction solution further include a second group of reaction solution, and the detection method further includes: introducing the reaction wash solution into the detection chip, lowering the temperature of the detection chip to 3° C.-6° C., for example, 4°° C. or 5° C., etc., and taking a fluorescence image; introducing the second group of reaction solution into the detection chip, raising the temperature of the detection chip to 60° C.-70° C., for example, 65° C., etc.; and taking a fluorescence image.

For example, in some embodiments, the reaction wash solution includes Tris-HCl (2-chloro-1,3-dimethylimidazolium hexafluorophosphate), (NH₄)₂SO₄, KCl, MgSO₄ and TWEEN ®20. For example, in some examples, the reaction wash solution includes 20 m mol/L Tris-HCl (pH 8.8), 10 m mol/L (NH₄)₂SO₄, 50 m mol/L KCl, 2 m mol/L MgSO₄ and 0.1% Tween®20.

For example, in some embodiments, the reaction mother solution includes Tris-HCl, (NH₄)₂SO₄, KCl, MgSO₄, Tween®20, enzyme and CIP. For example, in some examples, the reaction mother solution includes 20 m mol/L Tris-HCl (pH 8.8), 10 m mol/L (NH₄)₂SO₄, 50 m mol/L KCl, 2 m mol/L MgSO₄, 0.1% Tween®20, 8000 unit/mL BST-DNA polymerase (Bst polymerase) and 100 unit/mL CIP (2-chloro-1,3-dimethylimidazolium hexafluorophosphate).

For example, in some embodiments, the prepared reaction solution include six groups as illustrated in Table 1.

TABLE 1

| Group | Solution Preparation |
|---|---|
| 1A | reaction mother solution + |
| | 20 μmol/L dATP-TG + |
| | 20 μmol/L dCTP-TG |
| 1B | reaction mother solution + |
| | 20 μmol/L dGTP-TG + |
| | 20 μmol/L dTTP-TG |
| 2A | reaction mother solution + |
| | 20 μmol/L dATP-TG + |
| | 20 μmol/L dGTP-TG |
| 2B | reaction mother solution + |
| | 20 μmol/L dCTP-TG + |
| | 20 μmol/L dTTP-TG |
| 3A | reaction mother solution + |
| | 20 μmol/L dATP-TG + |
| | 20 μmol/L dTTP-TG |
| 3B | reaction mother solution + |
| | 20 μmol/L dCTP-TG + |
| | 20 μmol/L dGTP-TG |

In Table 1, dATP-TG is phosphate-labeled fluorescent deoxyadenine nucleotide; dTTP-TG is phosphate-labeled fluorescent deoxythymidine nucleotide; dCTP-TG is phosphate-labeled fluorescent deoxycytosine nucleotide; dGTP-CG is phosphate-labeled fluorescent deoxyguanine nucleotide, and these are the raw materials for fluorescent labels for DNA sequencing.

For example, during the detection process, the above-mentioned groups of reaction solution are sequentially used to perform the above-mentioned detection steps, and the DNA sequence is determined according to the fluorescence images.

For example, the above-mentioned detection results are used in fields such as early cancer screening, to obtain methylation site analysis results, and to distinguish normal and different cancer subtype samples to the greatest extent.

The following statements should be noted:
(1) The drawings involve only the structure(s) in connection with the embodiment(s) of the present disclosure, and other structure(s) can be referred to common design(s).
(2) For clarity, in the drawings used to describe the embodiments of the present disclosure, the thicknesses of layers or regions are enlarged or reduced, that is, the drawings are not drawn to actual scale. It can be understood that when a component such as a layer, film, region or substrate is referred to as being “on” or “under” another component, the component may be “directly” “on” or “under” another component, or one or more intermediate components may be interposed therebetween.
(3) In case of no conflict, features in one embodiment or in different embodiments can be combined to obtain new embodiments.

What have been described above are only specific implementations of the present disclosure, the protection scope of the present disclosure is not limited thereto, and the protection scope of the present disclosure should be based on the protection scope of the claims.

1. A detection chip, comprising:
a base substrate, and
a detection layer, provided on the base substrate and comprising a plurality of detection holes, wherein
an inner wall of each of at least part of detection holes among the plurality of detection holes is hydrophilic, and a contact angle of the inner wall is within 30 degrees.

2. The detection chip according to claim 1, wherein
a surface of the detection layer far away from the base substrate is hydrophobic relative to the inner wall of the at least part of detection holes, and a contact angle of the surface is 80-150 degrees, or
the surface of the detection layer far away from the base substrate is provided with a microstructure, and a contact angle of at least a surface of the microstructure far away from the base substrate is 80-150 degrees, wherein a surface roughness Ra of the microstructure is 250 nm-300 nm.

3. (canceled)

4. The detection chip according to claim 2, wherein the microstructure comprises a carbonized adhesive layer or an ITO layer doped with ZnO, and the detection layer comprises a silicon oxide layer.

5. (canceled)

6. The detection chip according to claim 1, wherein
a diameter of each detection hole among the plurality of detection holes is 0.2 microns-3.0 microns,
a distance between adjacent detection holes is 0.5 microns-2.5 microns,
a distance between centers of adjacent detection holes is 0.8 microns-5.0 microns, and
a depth of each detection hole among the plurality of detection holes is 0.5 microns-3.0 microns.

7. (canceled)

8. The detection chip according to claim 1, wherein a surface of the detection layer far away from the base substrate and/or the inner wall of at least one detection hole among the plurality of detection holes are provided with a metal element.

9. The detection chip according to claim 1, wherein
the inner wall of each among the plurality of detection holes comprises a side wall and a bottom wall, and
at least one of the side wall and the bottom wall is provided with an uneven structure.

10. The detection chip according to claim 1, wherein
a cross-sectional shape, parallel to the base substrate, of at least one detection hole among the plurality of detection holes is a circle, and
a cross-sectional shape, perpendicular to the base substrate, of at least one detection hole among the plurality of detection holes is a rectangle or a trapezoid.

11. The detection chip according to claim 10, wherein
a surface of the detection layer far away from the base substrate is provided with a microstructure, and the microstructure comprises a base layer and a hydrophobic layer provided on a side of the base layer far away from the detection layer; and
the base layer is an ITO layer, and the hydrophobic layer is an ITO layer doped with ZnO.

12. The detection chip according to claim 11, wherein
a diameter of each detection hole among the plurality of detection holes is 2.0 microns-3.0 microns, and
a distance between adjacent detection holes is 0.5 microns-1.5 microns, and
a depth of each detection hole among the plurality of detection holes is 0.5 microns-1.5 microns, and
a thickness of the microstructure is 0.2 microns-0.8 microns.

13. (canceled)

14. The detection chip according to claim 10, wherein
a surface of the detection layer far away from the base substrate is provided with a microstructure, and the microstructure comprises a base layer and a hydrophobic layer provided on a side of the base layer far away from the detection layer, and
the base layer is a metal layer, and the hydrophobic layer is a carbonized adhesive layer.

15. (canceled)

16. The detection chip according to claim 14, wherein the hydrophobic layer comprises a plurality of annular portions respectively surrounding the plurality of detection holes, the plurality of annular portions form a concave portion between every four adjacent detection holes, and a material of the hydrophobic layer does not exist in the concave portion.

17. The detection chip according to claim 14, wherein
a diameter of each detection hole among the plurality of detection holes is 1.3 microns-2.5 microns,
a distance between adjacent detection holes is 0.5 microns-2.0 microns,
a depth of each detection hole among the plurality of detection holes is 0.5 microns-1.5 microns, and
a thickness of the microstructure is 0.2 microns-1.0 micron.

18. (canceled)

19. The detection chip according to claim 1, further comprising:
a base adhesive layer, provided between the base substrate and the detection layer;
a first protective layer, comprising silicon dioxide and provided between the base adhesive layer and the detection layer, and
a second protective layer, comprising silicon dioxide and provided on a side of the detection layer far away from the base substrate,
wherein the detection layer comprises a photoresist material.

20. (canceled)

21. The detection chip according to claim 19, wherein
a diameter of each detection hole among the plurality of detection holes is 0.2 microns-2.2 microns, and
a depth of each detection hole among the plurality of detection holes is 0.5 microns-1.5 microns.

22. The detection chip according to claim 19, wherein a cross-sectional shape, perpendicular to the base substrate, of at least one detection hole among the plurality of detection holes comprises two arc-shaped edges protruding toward each other.

23. The detection chip according to claim 22, wherein
a first cross-section, parallel to the base substrate, of the at least one detection hole has a first diameter, a second cross-section, parallel to the base substrate, of the at least one detection hole has a second diameter, and a third cross-section, parallel to the base substrate, of the at least one detection hole has a third diameter,
the second cross-section is located on a side of the first cross-section far away from the base substrate, and the third cross-section is located on a side of the second cross-section far away from the base substrate, and
the second diameter is smaller than the first diameter and the third diameter.

24. The detection chip according to claim 1, wherein
the detection layer comprises a first sub-detection layer and a second sub-detection layer provided on a side of the first sub-detection layer far away from the base substrate, the first sub-detection layer is an adhesive layer, and the second sub-detection layer is a silicon oxide layer, and
the detection hole comprises a first sub-detection hole provided in the first sub-detection layer and a second sub-detection hole provided in the second sub-detection layer, and the first sub-detection hole and the second sub-detection hole are interpenetrated with each other, and
an orthographic projection of the second sub-detection hole on the base substrate is located inside an orthographic projection of the first sub-detection hole on the base substrate.

25. (canceled)

26. The detection chip according to claim 24, wherein
a diameter of the first sub-detection hole is 1.0 micron-2.5 microns, and a depth of the first sub-detection hole is 1.0 micron-1.8 microns, and
a diameter of the second sub-detection hole is 0.6 microns-1.8 microns, and a depth of the second sub-detection hole is 0.4 microns-0.8 microns.

27. The detection chip according to claim 1, wherein
a cross-sectional shape, parallel to the base substrate, of each of the at least part of detection holes among the plurality of detection holes is a circle,
a cross-sectional shape, perpendicular to the base substrate, of each of at least part of detection holes among the plurality of detection holes is an inverted trapezoid, and
a length of a long side of the inverted trapezoid is 1.2 microns-2.2 microns, a length of a short side of the inverted trapezoid is 0.5 microns-1.8 microns, and a height of the inverted trapezoid is 1.0 micron-1.8 microns.

28. (canceled)

29. The detection chip according to claim 1, wherein
a cross-sectional shape, parallel to the base substrate, of each of the at least part of detection holes among the plurality of detection holes is a circle,
a cross-sectional shape, perpendicular to the base substrate, of each of the at least part of detection holes among the plurality of detection holes is a rectangle,
a diameter of each of the at least part of detection holes among the plurality of detection holes is 1.0 micron-2.2 microns, and
a depth of each of the at least part of detection holes among the plurality of detection holes is 1.0 micron-1.8 microns.

30-38. (canceled)
